(19) United States
(12) Patent Application Publication
(10) Pub. No.: US 2019/0389958 A1
(43) Pub. Date: Dec. 26, 2019

(54) ANTI-CXCR4 ANTIBODIES

(71) Applicant: MSM Protein Technologies, Inc., East Kingston, NH (US)

(72) Inventors: Andres McAllister, Celigny (CH); Corinne Moulton, Sainte-legier (CH); Colleen Stauffer-Kelleher, Epalinges (CH); Svetlana Abbasova, Petushinski District (RU); Viktoriia Vasilyeva, East Princeton, NJ (US); Olga Rimkevich, Beverly, MA (US); Andrey Ulitin, Moscow (RU); Roman Mikhaylov, Moskovskaya Oblast (RU); Tajib Mirzabekov, Watertown, MA (US); David Kreimer, Berkeley, CA (US); Eldar Kim, Belmont, MA (US)

(73) Assignee: MSM Protein Technologies, Inc., East Kingston, NH (US)

(21) Appl. No.: 16/481,148

(22) PCT Filed: Jan. 31, 2017
(86) PCT No.: PCT/US17/15821

§ 371 Date: Jul. 26, 2019

(51) Int. Cl. C07K 16/28; G01N 33/574; A61P 35/00; A61P 35/04

(52) U.S. Cl. CPC C07K 16/2866; G01N 33/57415; A61P 35/00; A61P 35/04; C07K 2317/732; C07K 2317/21; C07K 2317/55; C07K 2317/92; C07K 2317/76; C07K 2317/565

(57) ABSTRACT

The present invention relates to monospecific antibodies against CXCR4 or binding fragments thereof, to the use of such anti-CXCR4 antibodies or binding fragments in treating diseases whose pathogenesis is related to activation of CXCR4, as well as to pharmaceutical compositions comprising such anti-CXCR4 antibodies or binding fragments.

### FIELD OF THE INVENTION

The present invention relates to monospecific anti-CXCR4 antibodies and binding fragments, to the use of such anti-CXCR4 antibodies and binding fragments in treating diseases whose pathogenesis is related to activation of CXCR4 as well as to pharmaceutical compositions and kits comprising such anti-CXCR4 antibodies and binding fragments.

### BACKGROUND

G protein-coupled receptors (GPCRs), also known as seven-transmembrane domain receptors, form a superfamily of proteins that generally play important roles in a variety of biological and pathological processes. Chemokine receptors represent a sub-family of GPCRs, which are named after the ability of their ligands (i.e. chemokines) to induce directed chemotaxis in nearby responsive cells. Among these chemokine receptors, CXCR4 (also known in the art as, for example, LESTR, Fusin or CD 184) plays an important role in immune and inflammatory responses by mediating the directional migration and activation of leukocytes. CXCR4 has also been shown to be expressed or over-expressed in a variety of cancer cell lines and tissues. An important ligand of CXCR4 is stromal cell-derived factor-1 (SDF-1, also known as CXCL12). The CXCR4 and SDF-1 interaction seems to play an important role in multiple phases of tumorigenesis, including tumor growth, invasion, angiogenesis, and metastasis. Ubiquitin is another known ligand of CXCR4.

Several CXCR4 antagonists have been identified and/or developed in view of treating diseases related to CXCR4 activation. For example, plerixafor or AMD3100, a bicyclam CXCR4 antagonist, is FDA approved for use in combination with granulocyte colony-stimulating factor to mobilize hematopoietic stem cells to the bloodstream for collection and subsequent autologous transplantation in patients with multiple myeloma and non-Hodgkins lymphoma. LY2510924, a CXCR4 antagonist peptide, is currently in Phase II clinical trials for cancer.

An example of a known anti-CXCR4 antibody is 12G5, a mouse antibody commonly used as a reagent/positive control in lab experiments.

Although there are several agents, either available or under development, that target CXCR4, there still remains a need for effective therapeutic agents targeting CXCR4.

### SUMMARY OF THE INVENTION

The present invention relates to a monospecific antibody or binding fragment thereof, comprising a light chain variable region having CDR1L, CDR2L and CDR3L and a heavy chain variable region having CDR1H, CDR2H and CDR3H, wherein said CDR1L comprises the amino acid sequence SEQ ID NO: 1, said CDR2L comprises the amino acid sequence SEQ ID NO: 2, said CDR3L comprises the amino acid sequence SEQ ID NO: 3 or 4, said CDR1H comprises the amino acid sequence SEQ ID NO: 5 or 6, said CDR2H comprises the amino acid sequence SEQ ID NO: 7, 8, 9 or 10, and said CDR3H comprises the amino acid sequence SEQ ID NO: 11 or 12. Also encompassed are variants of the sequences of SEQ ID NOs: 1-12 that contain one or more conservative modifications. The antibody or binding fragment specifically binds to human CXCR4.

The present invention further relates to a monospecific antibody or binding fragment thereof, comprising a light chain variable region comprising the amino acid sequence of SEQ ID NO: 13 or 14. Also encompassed are variants of the latter sequences that contain conservative modifications. The monospecific antibody or binding fragment further comprises a heavy chain variable region comprising the amino acid sequence of SEQ ID NO: 15, 16, 17, 18 or 19. Also encompassed are variants of the latter sequences that contain one or more conservative modifications. The antibody or binding fragment specifically binds to human CXCR4.

In a specific embodiment, the afore-mentioned monospecific antibodies or binding fragments of the invention are human engineered antibodies or binding fragments, respectively.

In a more specific embodiment, the afore-mentioned monospecific antibodies of the invention are of the IgG isotype.

The present invention further encompasses therapeutic as well as diagnostic applications of the monospecific antibodies and binding fragments of the invention.

In an in vitro diagnostic assay to discover CXCR4-expressing cancer cells in a human or another mammalian subject, a biopsy or fluid sample containing cancer cells taken from the subject can be analysed in an immunochemical or immunohistochemical assay that employs a monospecific antibody or binding fragment of the invention to detect CXCR4. An in vivo diagnostic assay to discover CXCR4-expressing cancer cells and tissues in a human or other mammalian subject can make use of a monospecific antibody or binding fragment of the invention that has been radioactively labelled. In the assay, the radiolabelled antibody or binding fragment is administered, typically parenterally, to the subject, and the distribution of the antibody or binding fragment is assessed subsequently by immunoscintigraphy.

The present invention further relates to methods of treating cancers expressing CXCR4 including Burkitt's lymphoma and breast cancers, comprising administering a therapeutically effective amount of a monospecific antibody or binding fragment of the invention to a human or other mammalian subject in need of such treatment. The present invention further relates to the use of a monospecific antibody or binding fragment of the invention for treatment of a cancer expressing CXCR4 including Burkitt's lymphoma and breast cancers.

The present invention further relates to methods of preventing metastasis of breast cancer or another cancer expressing CXCR4, comprising administering a therapeutically effective amount of a monospecific antibody or binding fragment of the invention to a human or nonhuman mammalian subject in need of such treatment. The present invention further relates to the use of a monospecific antibody or binding fragment of the invention for prevention of metastasis of breast cancer or other cancers expressing CXCR4.

The present invention also relates to pharmaceutical compositions comprising a therapeutically effective amount of a monospecific antibody or binding fragment of the invention and a pharmaceutically acceptable excipient. Typically, such pharmaceutical compositions are for parenteral administration to a subject and, therefore, comprise a therapeutically effective amount of a monospecific antibody or binding fragment of the invention, a parenterally acceptable diluent and, optionally, a pharmaceutically acceptable excipient. Also encompassed are diagnostic kits comprising a monospecific antibody or binding fragment of the invention.

The invention also concerns isolated polynucleotides encoding a monospecific antibody or binding fragment of the invention. Thus, it also relates to a polynucleotide (or isolated polynucleotide) encoding a monospecific antibody or binding fragment thereof, comprising a light chain variable region having CDR1L, CDR2L and CDR3L and a heavy chain variable region having CDR1H, CDR2H and CDR3H, wherein said CDR1L comprises the amino acid sequence SEQ ID NO: 1, said CDR2L comprises the amino acid sequence SEQ ID NO: 2, said CDR3L comprises the amino acid sequence SEQ ID NO: 3 or 4, said CDR1H comprises the amino acid sequence SEQ ID NO: 5 or 6, said CDR2H comprises the amino acid sequence SEQ ID NO: 7, 8, 9 or 10, and said CDR3H comprises the amino acid sequence SEQ ID NO: 11 or 12. Also encompassed are variants of the sequences of SEQ ID NOs: 1-12 that contain one or more conservative modifications. The antibody or binding fragment expressed from the latter polynucleotides specifically binds to human CXCR4. For example, the polynucleotide can comprise the CDR1L-encoding polynucleotide of SEQ ID NO: 20, the CDR2L-encoding polynucleotide of SEQ ID NO: 21, the CDR3L-encoding polynucleotide of SEQ ID NO: 22 or 23, the CDR1H-encoding polynucleotide of SEQ ID NO: 24 or 25, the CDR2H-encoding polynucleotide of SEQ ID NO: 26, 27, 28 or 29, and the CDR3H-encoding polynucleotide of SEQ ID NO: 30 or 31.

More specifically, a polynucleotide (or isolated polynucleotide) encoding a monospecific antibody or binding fragment thereof can comprise a light chain variable region comprising the amino acid sequence of SEQ ID NO: 13 or 14. Also encompassed are variants of the latter sequences that contain conservative modifications. The monospecific antibody or binding fragment further comprises a heavy chain variable region comprising the amino acid sequence SEQ ID NO: 15, 16, 17, 18 or 19. Also encompassed are variants of the latter sequences that contain one or more conservative modifications. The antibody or binding fragment expressed from the latter polynucleotide specifically binds to human CXCR4. For example, the polynucleotide can comprise the light chain variable region-encoding polynucleotide of SEQ ID NO: 32 or 33 and the heavy chain variable region-encoding polynucleotide of SEQ ID NO: 34, 35, 36, 37 or 38.

### BRIEF DESCRIPTION OF THE DRAWINGS

FIG. 1 represents dose response curves of CXCR4 binding for antibodies of the present invention in IgG1 format, obtained as described under Example 4. FIGS. 1a to 1e represent dose response curves for V62.1, V62.1-R108H, V62.1-H-m80, V62.1-H-m43-m38 and V62.1-H-m47-m38, respectively.

FIG. 2 represents in vivo luciferase activities in the breast region of mice as measured in the anti-metastatic model of Example 12.

### DETAILED DESCRIPTION OF THE INVENTION

The present invention relates to anti-CXCR4 antibodies and uses thereof as well as to pharmaceutical compositions comprising anti-CXCR4 antibodies.

So that the invention may be more readily understood, certain terms are specifically defined below. Unless explicitly defined elsewhere in this document, all other technical and scientific terms used herein have the meaning that would be commonly understood by one of ordinary skill in the relevant art.

As used herein, including in the appended claims, the singular forms of words such as “a”, “an”, and “the”, include their corresponding plural references unless the context clearly indicated otherwise.

The term “human CXCR4” refers to a protein whose amino acid sequence is at least 90%, at least 95%, or at least 96%, 97%, 98%, or 99% identical to the complete amino acid sequence of human CXCR4 having Genbank accession number P61073, or to a protein that has substantially the same biological function as CXCR4 but whose sequence differs from the complete amino acid sequence of human CXCR4 by the substitution, insertion or deletion of one or more amino acids.

The general structure of an “antibody” is well-known in the art. For an antibody of the IgG type, there are four amino acid chains (two “heavy” chains and two “light” chains) that are cross-linked via inter-chain disulfide bonds. Each of the heavy and light chains has a variable N-terminal region and a constant region. The constant regions of an immunoglobulin antibody are called the Fc portion and are highly conserved in humans. The variable regions of each light/heavy chain pair form a variable domain that comprises the antibody's antigen binding site.

Each of the heavy and light chain variable regions can be further subdivided into regions of hypervariability, named complementarity determining regions (CDRs) that are interspersed with regions that are more conserved, named framework regions (FR). Each variable region is composed of three CDRs and four FRs that are arranged from amino-terminus to carboxy-terminus in the following order: FR1, CDR1, FR2, CDR2, FR3, CDR3, FR4. Herein, the three CDRs of the heavy chain are referred to as CDR1H, CDR2H, and CDR3H and the three CDRs of the light chain are referred to as CDR1L, CDR2L and CDR3L. The CDRs contain most of the residues that form specific interactions with the antigen. In the following, the heavy and light chain variable regions may be respectively referred to as HCVR and LCVR.

As used herein, the term “conservative modifications” of a given amino acid sequence of an antibody or a binding fragment, or of parts thereof, refers to amino acid modifications that do not significantly affect or alter the binding characteristics of the antibody, binding fragment, or parts thereof, containing the amino acid sequence. Such conservative modifications include amino acid substitutions, additions and deletions. Modifications can be introduced into an antibody of this disclosure by standard techniques known in the art, such as site-directed mutagenesis and PCR-mediated mutagenesis. Conservative amino acid substitutions are ones in which the amino acid residue is replaced with an amino acid residue having a side chain of related chemical character. Families of amino acid residues having side chains of related chemical character have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan,). Thus, one or more amino acid residues within the CDR regions of an antibody of this disclosure can be replaced with other amino acid residues from the same side chain family and the altered antibody can be tested for retained antigen-binding properties using the functional assays described herein.

The sequence numbering used herein follows Kabat et al. (1991) Sequences of proteins of immunological interest. Public Health Service, National Institutes of Health, Bethesda. The CDR definitions used herein follow the method described in MacCallum et al. (1996) J. Mol. Biol. 626:732-745.

An antibody according to the present invention can be intact, comprising complete or full length constant regions, including the Fc region, or a portion or fragment of such an antibody (“binding fragment”) that comprises the antigen-binding portion and retains antigen-binding capability. Such a portion or fragment can include, e.g., a Fab fragment (“fragment antigen binding”; i.e. the region of an antibody that binds to antigens) that is composed of a pair of heavy and light chain fragments each containing a constant and a variable region, or a Fab′ or F(ab′)2 fragment that includes the CDRs or the variable regions of the anti-CXCR4 antibodies disclosed herein. Furthermore, such a portion or fragment can be a single chain Fv fragment that may be produced from a polynucleotide comprising nucleotide sequences encoding light and heavy chain variable regions, whereby the latter nucleotide sequences are separated by a linker sequence (e.g., Pluckthun, The Pharmacology of Monoclonal Antibodies, vol. 113, Rosenburg and Moore eds., Springer-Verlag, New York, pp 269-315, 1994). Regardless of whether fragments or portions are specified, the term “binding fragment” as used herein includes such fragments or portions as well as single chain forms unless otherwise indicated. As long as a protein retains the ability to specifically or preferentially bind CXCR4 and includes a CDR sequence(s) disclosed herein, it is included in the terms “antibody” and “binding fragment”, respectively. It is understood that only full length antibodies may perform certain effector functions such as Antibody Dependent Cell Cytotoxicity (ADCC).

Antibodies of the present invention may have a heavy chain constant region selected from any of the immunoglobulin classes (IgA, IgD, IgG, IgM, and IgE). Preferably, antibodies of the present invention are of the IgG type, more preferably the IgG1 isotype. It is to be understood that, unless there is an indication to the contrary, the term “IgG1” in the present text refers to human IgG1.

The term “human engineered antibody” refers to an antibody having frameworks, hinge regions, and constant regions of human origin that are identical with or substantially identical (substantially human) with frameworks, hinge regions and constant regions derived from human genomic sequences. Fully human frameworks, hinge regions, and constant regions encompass sequences expressed in the human germline as well as sequences containing spontaneous somatic mutations. A human engineered antibody may comprise framework, hinge, or constant regions derived from fully human framework, hinge, or constant regions containing one or more amino acid substitutions, deletions, or additions therein, and/or glycosylation modifications. A “human engineered binding fragment” refers to a portion or fragment of a human engineered antibody. Often, a human engineered antibody is substantially non-immunogenic in humans.

A variety of different human framework sequences may be used singly or in combination as a basis for the human engineered antibodies of the present invention. Preferably, the framework regions of the antibodies of the invention are of human origin or substantially human (at least 95%, 97% or 99% of human origin). The sequences of framework regions of human origin may be obtained from Current Trends in Monoclonal Antibody Development and Manufacturing by Shire et al., ISBN 978-O-387-76643-0. Preferably, in antibodies according to the present invention, the framework region of the heavy chain corresponds to the germline consensus sequence subgroup Ill. Preferably also, in antibodies according to the present invention, the framework region of the light chain corresponds to the germline kappa Ill consensus sequence.

As used herein, the terms “monospecific antibody” or “monospecific antibody composition” refer to a preparation of antibody molecules having identical protein sequences (ionic or oxidation microvariants being included). A monospecific antibody composition displays a single binding specificity and affinity for a particular epitope.

As used herein, an antibody that “specifically binds to human CXCR4” refers to an antibody that binds to human CXCR4 (and possibly CXCR4 from one or more non-human species) with an EC50 of 50 nM or less, as measured in a Fluorescent Flow Cytometry-based assay as described in Example 4 herein below, but does not substantially bind to other GPCRs such as, for example, CXCR7.

As used herein when referring to an antibody, the phrase “does not substantially bind” to non-CXCR4 proteins means that the antibody does not bind at all or exhibits only weak binding to non-CXCR4 proteins. The EC50 value for such weak binding can be equal to or greater than 100 nM as measured in a Fluorescent Flow Cytometry-based assay as described in Example 4.

As used herein, ADCC refers to Antibody Dependent Cell Cytotoxicity, i.e. antibody mediated cell death, which is an antibody effector function mainly prompted by the Fc region. Antibodies of IgG isotypes, particulary IgG1, are known for having good ADCC properties.

When referring to SDF-1 or CXCL12 herein, unless otherwise specified or exemplified, it is meant to designate any and all human SDF-1 variants, including e.g. SDF-1alpha or CXCL12a and SDF-1beta or CXCL12b.

When referring to the binding properties, half maximal Effective Concentration 50 (EC50) is the concentration which induces a response halfway between the baseline and the maximal binding of a given antibody. It is calculated via a dose response curve, as explained in Example 4 herein.

A “subject” is a mammal, preferably a human.

The term “treating” (or “treat” or “treatment”) means slowing, stopping, reducing, or reversing the progression or severity of a symptom, disorder, condition or disease.

The term “preventing” (or “prevent” or “prevention”) means prohibiting, restraining, or inhibiting the incidence, occurrence or recurrence of a symptom, disorder, condition, or disease.

The term “therapeutically effective amount” refers to the amount or dose of an antibody of the present invention which, upon single or multiple dose administration to a patient, provides the desired treatment.

Particular antibodies of the present invention originate from a phage display library, and from affinity maturation processes as described herein.

Phage-display libraries are commonly used technologies for selection of antibody fragments that provide a starting point for generation and optimization of human engineered antibodies. See e.g. Hoogenboom (2005) Nat. Biotechnol. 23: 1105-1116; Bradbury & Marks (2004) J. Immunol. Methods 290: 29-49; and Fredericks et al., (2004) Protein Eng. Des. Sel. 17: 95-106. Other types of display technologies useful for the generation and affinity maturation (optimization) including yeast-, mRNA- and ribosome-display libraries are gaining in popularity for selection and optimization of antibodies (see Hoogenboom, Bradbury & Marks, and Fredericks et al.).

Display libraries may display single-chain variable-domain antibody fragments (scFvs) or Fab fragments, and contain the encoding DNA or RNA. They have high genetic diversity or repertoire size (commonly 10\hat{ }9-10\hat{ }13). The genetic diversity in these libraries is commonly created by cloning the repertoire of the immunoglobulin heavy chain and light chain variable gene segments from naive or immunized individuals. Alternatively, this diversity can be achieved by randomization of CDR sequences, including using chemically synthesized CDR fragments, or by a combination of these two approaches. The binding step (for selections from such a library) can then be undertaken with the target (receptor) in solution, immobilized on a surface, on liposomes (such as proteoliposomes described in U.S. Pat. No. 6,761,902), on cells, etc. After extensive washing, bound clones are recovered and amplified for a further round of selection.

Affinity maturation processes may then be performed on initial best binder antibody candidates to try to obtain derivative candidates with improved properties, such as better stability and/or improved binding, etc. Several affinity maturation strategies are available to a person skilled in the art, such as, but not limited to, directed comprehensive mutagenesis, CDR or light/heavy chain shuffling, point insertion(s) or deletion(s) in CDRs, or any combination of these approaches.

Particular antibodies of the present invention include antibodies as disclosed in Examples 1 and 2 herein. It is to be understood that the present invention also embraces each and every possible exchange of CDRs between the variable regions provided herein. Preferably, a heavy chain CDR may be exchanged with another heavy chain variable region CDR, and likewise, a light chain CDR may be exchanged with another light chain variable region CDR.

### Antibody Synthesis

Antibodies of the invention can be produced using techniques well known in the art, e.g., recombinant technologies, in vitro protein expression technologies or combinations of such technologies or other technologies readily known in the art.

For example, Fab fragments obtained from a screen of a Fab display library directly or subsequent to affinity maturation can be converted into IgGs by commonly used techniques such as cloning into appropriate expression vectors encoding the desired constant region.

For direct production of an IgG antibody, an appropriate host cell, such as HEK 293 or CHO cells, may be either transiently or stably transduced with an expression system suitable for producing and secreting IgG antibodies. The expression system will comprise heavy chain and light chain expression constructs that are transduced at an optimized ratio or a single vector system comprising expressible light chain as well as heavy chain genes. Secreted antibody can be purified using any of many commonly-used techniques. For example, culture medium containing antibody can be conveniently applied to a Protein A or G Sepharose FF column that has been equilibrated with a compatible buffer, e.g., phosphate-buffered saline (pH 7.4). The column is then washed to remove non-specifically binding components. Bound antibody is eluted, for example, by application of a pH gradient. Antibody-containing fractions are detected, e.g., by SDS-PAGE, and are pooled. Depending on the intended use, the antibody can be further purified. The antibody can be concentrated and/or sterile-filtered using common techniques. Soluble aggregates and multimers can be effectively removed by common techniques, including size exclusion, hydrophobic interaction, ion exchange, or hydroxyapatite chromatography. Purified antibody typically can be stored refrigerated, frozen, or lyophilized.

The person of skill in the art will know that Fab antibodies can be similarly produced using cells such as bacterial, fungal (yeast) or insect cells.

### Properties of Antibodies of the Invention

The antibodies of the present invention, in Fab format and/or in IgG format, were characterized in respect of several desirable biological properties.

Binding to CXCR4 is the first criterion for efficacy of the antibodies according to the present invention. Antibodies according to the present invention specifically bind to CXCR4 with an EC50 of below 50 nM, preferably below 10 nM, as revealed by experiments using cells expressing CXCR4 from a transfected, expressible gene and/or tumor cell lines expressing CXCR4.

Conversion of Fab fragments into IgG antibodies generally improves receptor binding (EC50). This was also verified with antibodies of the present invention. Preferably, when in IgG1 format, antibodies according to the present invention specifically bind CXCR4 with an EC50 of below 5 nM.

The antibodies of the present invention inhibit binding of SDF-1 to the CXCR4 receptor and prevent receptor activation. Consequences of SDF-1 binding to its receptor include, for example, calcium flux induction and cell migration, which are important parameters for cancer cell invasion. The antibodies of the present invention inhibit calcium flux induction and/or migration of CXCR4-expressing cells.

As has been demonstrated for antibodies such as trastuzumab and rituximab, ADCC can be an important mechanism of action of therapeutic antibodies against tumors. The antibodies of the present invention were shown to be capable of ADCC. Studies with xenograft tumor models demonstrated the anti-tumor activity of the antibodies of the invention.

Pharmaceutical Compositions and their Administration

The present invention also concerns pharmaceutical compositions comprising an antibody of the present invention. The latter compositions will be preferentially administered parenterally, but transnasal, transpulmonary or transdermal delivery is also envisaged. The pharmaceutical compositions may contain any conventional non-toxic pharmaceutically-acceptable excipient and, in the case of a liquid formulation, diluent. In some cases, the pH of the formulation may be adjusted with pharmaceutically acceptable acids, bases or buffers to enhance the stability of the formulated agent or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion.

Antibody of the invention can be stored as a lyophilized formulation or as a solution. Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent. Among the acceptable diluents that may be employed are water, Ringer's solution, U.S.P. and isotonic sodium chloride solution. In addition, sterile, fixed oils are conventionally employed as a solvent or suspending medium. The compositions can further comprise “pharmaceutically-acceptable” excipients or stabilizers typically employed in the art (all of which are termed “excipients”). Excipients comprise, e.g., buffering agents, stabilizing agents, preservatives, tonicity agents, non-ionic detergents, antioxidants and other miscellaneous additives. (See Remington's Pharmaceutical Sciences, 16th edition, A. Osol, Ed. (1980)). Such additives must be nontoxic to the recipients at the dosages and concentrations employed.

Buffering agents are preferably present at concentration ranging from about 2 mM to about 50 mM. Suitable buffering agents include both organic and inorganic acids and salts thereof such as citrate buffers (e.g., monosodium citrate-disodium citrate mixture, citric acid-trisodium citrate mixture. citric acid-monosodium citrate mixture. etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, there may be the mentioned phosphate buffers, histidine buffers and trimethylamine salts such as Tris. Preservatives may be added to retard microbial growth, and may be added in amounts ranging from 0.2%-1% (w/v). Suitable preservatives for use with the present invention include phenol, benzyl alcohol, meta-cresol, methyl paraben, propyl paraben, octadecyldimethylbenzyl ammonium chloride, benzalconium halides (e.g., chloride, bromide, iodide), hexamethonium chloride, alkyl parabens such as methyl or propyl paraben, catechol, resorcinol, cyclohexanol, and 3-pentanol.

The osmolarity of the pharmaceutical compositions may be adjusted with tonicity agents to a value that is compatible with the intended use of the compositions. For example, the osmolality of injectable solutions may be adjusted to approximately the osmotic pressure of blood, which is equivalent to about 0.9 w/v % of sodium chloride in water. Examples of suitable tonicity agents include chloride salts of sodium, potassium, calcium and magnesium, dextrose, glycerol, propylene glycol, mannitol, sorbitol, erythritol, arabitol, xylitol, and the like and mixtures thereof. Tonicity agents are typically used in amounts ranging from about 0.001 to about 1% w/v. These amounts have been found to be useful in providing a physiologically acceptable tonicity. Preferably, the tonicity agent(s) will be employed in an amount to provide a final osmotic value to the composition of 150 to 450 mOsm/kg, more preferably between about 220 and about 350 mOsm/kg, and most preferably between about 270 and about 300 mOsm/kg.

The compositions can further comprise a stablilizer. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, alpha-monothioglycerol and sodium thiosulfate; low molecular weight polypeptides (i.e. <10 residues); proteins such as human serum albumin, bovine serum albumin, gelatin or immunoglobulins; hydrophylic polymers such as polyvinylpyrrolidone; monosaccharides such as xylose, mannose, fructose, glucose; disaccharides such as lactose, maltose, sucrose and trisaccacharides such as raffinose; polysaccharides such as dextran. Stabilizers may be present in the weight range from 0.1 to 10,000 times the weight of the antibody of the invention.

Wetting agents may be added to help solubilize the antibody of the invention as well as to protect it against agitation-induced aggregation. Suitable wetting agents include non-ionic surfactants such as polysorbates (20, 80, etc.), polyoxamers (184, 188 etc.), Pluronic®, polyols, polyoxyethylene sorbitan monoethers (Tween®-20, Tween®-80, etc.). Non-ionic surfactants may be present in a range of about 0.05 mg/ml to about 1.0 mg/ml, preferably about 0.07 mg/ml to about 0.2 mg/ml.

The pharmaceutical compositions may also contain an additional active compound as necessary for the particular indication being treated, preferably a compound with an activity that does not adversely affect that of the antibody of the invention. For example, when a cancer is being treated, it may be desirable to further provide one or more chemotherapeutic agents. Such compounds are suitably present in combination in amounts that are effective for the purpose intended.

The pharmaceutical compositions can be sterilized, for example, by filtration through sterile filtration membranes.

Antibody of the invention may also be entrapped in microcapsules prepared, for example, by coacervation techniques or by interfacial polymerization, for example, hydroxymethylcellulose or gelatin-microcapsule and poly-(methylmethacylate) microcapsules, respectively, in colloidal drug delivery systems (for example, liposomes, albumin micropheres, microemulsions, nano-particles and nanocapsules) or in macroemulsions. Such techniques are disclosed in Remington's Pharmaceutical Sciences, 16th edition, A. Osal, Ed. (1980).

Sustained-release preparations may be prepared. Suitable examples of sustained-release preparations that may be adapted for the delivery of antibody of the invention include semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thiol-disulfide interchange, stabilization may be achieved by modifying sulfhydryl residues, lyophilizing from acidic solutions, controlling moisture content, using appropriate additives, and developing specific polymer matrix compositions.

Administration methods can be appropriately selected in consideration of a subject's age and symptoms. The dose in a pharmaceutical composition of antibody or binding fragment of the invention may be, for example, from about 0.0005 to about 100 mg/kg for each administration. More preferably, the dose may be from about 0.1 to about 20 mg/kg for each administration. Administration may be several times daily, daily, every two days, half-weekly or weekly. However, the present invention is not limited by the numeric values described above. The doses and administration methods vary depending on the subject's weight, age, symptoms, and such. Those skilled in the art can set appropriate doses and administration methods in consideration of the factors described above.

### Diagnostic Uses for the Antibodies of the Invention

The antibodies and binding fragments of the present invention can be useful in diagnostic assays, e.g., assays for detecting expression of CXCR4 on specific cells, tissues, or serum. For diagnostic applications, the antibody typically will be labeled with a detectable moiety. Numerous labels are available. Examples of enzymatic labels include luciferases (e.g., firefly luciferase and bacterial luciferase; U.S. Pat. No. 4,737,456), malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzym. (Ed. Langone & Van Vunakis), Academic press, New York, 73: 147-166 (1981).

Sometimes, the label is indirectly conjugated with the antibody. The skilled artisan will be aware of various techniques for achieving this. For example, the antibody can be conjugated with biotin and any of the labels mentioned above can be conjugated with avidin, or vice versa. Biotin binds selectively to avidin and thus, the label can be conjugated with the antibody variant in this indirect manner. Alternatively, to achieve indirect conjugation of the label with the antibody, the antibody is conjugated with a small hapten (e.g. digoxin) and one of the different types of labels mentioned above is conjugated with an anti-hapten antibody (e.g. anti-digoxin antibody). Thus, indirect conjugation of the label with the antibody can be achieved.

In another embodiment of the invention, the antibody of the invention need not be labeled, and the presence thereof can be detected using a labeled antibody which binds to the antibody.

The antibodies or binding fragment of the present invention may be employed in any known immunochemical assay method, such as competitive binding assays, direct and indirect sandwich assays, and immunoprecipitation assays. Zola, Monoclonal Antibodies: A Manual of Techniques, pp. 147-158 (CRC Press, Inc. 1987). They can also be used for immunohistochemical detection of CXCR4 on cells and tissues. For immunohistochemistry, a tissue sample, e.g., a tumor tissue sample, may be fresh or frozen or may be embedded in paraffin and fixed with a preservative such as formalin, for example.

The antibodies may also be used for in vivo diagnostic assays. Generally, the antibody or binding fragment is labeled with a radionucleotide (such as ¹¹¹In, ⁹⁹Tc, ¹⁴C, ¹³¹I, ³H, ³²P or ³⁵S) so that CXCR4-over-expressing cells can be localized using immunoscintiography.

The antibody or binding fragment of the present invention can be provided in a kit, i.e., a packaged combination of reagents in predetermined amounts with instructions for performing the diagnostic assay. Where the antibody is labeled with an enzyme, the kit may include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

### Human Therapeutic Uses for the Antibodies and Binding Fragments of the Invention

The antibodies of the invention can be used in stem cell and regenerative medicine. Interaction of CXCR4 with SDF-1alpha is important in holding hematopoietic stem cells in the bone marrow. Anti-CXCR4 antibodies can serve as antagonists that are capable of mobilizing hematopoietic stem cells into the bloodstream as peripheral blood stem cells. Peripheral blood stem cell mobilization can be important in hematopoietic stem cell transplantation (as an alternative to transplantation of surgically-harvested bone marrow) and is currently performed using drugs such as G-CSF. Antibodies and binding fragments of the present invention can also be used to prevent late stage HIV (X4 viruses) from interacting with the CXCR4 receptor and entering T cells.

The antibodies and binding fragments of the invention further can be used in the treatment of a variety of different cancers that express CXCR4. CXCR4 may be the chemokine receptor that is most commonly found on tumor cells, both in human and experimental murine cancers. The receptor has been found on at least the following tumor types: B-CLL, AML, B-lineage ALL (including Burkitt's lymphoma), follicular center myeloma, CML, multiple myeloma, pancreatic cancer, prostate cancer, breast cancer, ovarian cancer, thyroid cancer, colorectal cancer, oral squamous carcinoma, cervical cancer, neuroblastoma, kidney cancer, glioma, rhabdomyosarcoma, small lung cancer and melanoma. Balkwill (2004) Seminars in Cancer Biology 14: 171-9. Treatment will involve administration to the cancer patients of a pharmaceutical composition comprising an antibody or binding fragment of the invention. The composition may be administered by any suitable means, including parenteral, subcutaneous, intraperitoneal, intrapulmonary, intranasal, and intralesional administration. Parenteral infusions include intramuscular, intravenous, intraarterial, intraperitoneal, or subcutaneous administration. In addition, the antibody or binding fragment is suitably administered by pulse infusion, particularly with declining doses of the antibody or binding fragment. Preferably, the dosing is given by injections, most preferably intravenous or subcutaneous injections, depending in part on whether the administration is brief or chronic.

Depending on the type and severity of the disease, about 0.1 mg/kg to about 20 mg/kg of antibody or binding fragment is an initial candidate dosage for administration to the subject, whether, for example, by one or more separate administrations, or by continuous infusion. A typical daily dosage might range from about 1 mg/kg to 100 mg/kg or more.

The pharmaceutical composition comprising antibody or binding fragment of the invention will be formulated, dosed and administered in a manner consistent with good medical practice. Factors for consideration in this context include the type and stage of cancer, the clinical condition of the individual subject, the site of delivery of the agent, the method of administration, the scheduling of administration, and other factors known to medical practitioners. The “therapeutically effective amount” of the antibody to be administered will be governed by such considerations, and is the minimum amount necessary to treat the disease. The antibody need not be, but is optionally formulated with one or more agents currently used to treat the disease, e.g., one or more chemotherapeutic agents. The effective amount of such other agents depends on the amount of antibody or binding fragment present in the formulation, the type and stage of cancer, and other factors discussed above. These are generally used in the same dosages and with administration routes as they are currently used (without antibody of the invention) or from about 1 to 99% of the currently employed dosages.

### EXAMPLES

### Example 1: Preparation of Fab Phage Library and Screening of Phage Antibodies

The antibodies of the present invention were originally derived from a Fab library of the size of 10′11 comprised of pSF1 phagemids carrying Fab E. coli codon-optimized synthetic genes encoding human Fab heavy and human Fab light chains with randomized CDRs. For the heavy chain, the framework DP47 was employed, and for the light chain, the framework DPK22 was employed.

The phage library was generated employing protocols and CDR randomization schemes as described in Knappik et al. (2000) J. Mol. Biol. 296:57-86; Lee et al. (2004) J. Mol. Biol. 340:1073-93; Hoet et al. (2005) 23:344-8.

More specifically, in the Fab library the heavy chain CDR1, CDR2, and CDR3 and the light chain CDR3 were subjected to randomization. For the randomization of heavy chain CDR3, tri-nucleotide based oligonucleotides were employed as described in Knappik et al., whereas for other CDRs standard nucleotide mixtures were employed to generate CDR oligonucleotides.

The common light chain CDRs of the Fab library were as follows (MacCallum et al. (1996) J. Mol. Biol. 626:732-745):

| (SEQ ID No. 1) | |
|---|---|
| CDR1L | SSYLAWY |
| (SEQ ID No. 2) | |
| CDR2L | LLIYGASSRA |

For the screening of the Fab library, a Magnetic ProteoLiposome technology was used in order to display CXCR4 in a liposome membrane in a conformation closely resembling its native conformation. See Mirzabekov et al. (2000) Nat Biotechnol. 18:649-54 and U.S. Pat. No. 6,761,902.

Screening of the Fab library was carried out using methods described by Mirzabekov et al. and yielded the following Fab candidate:

| | SEQ ID NO. | | | | | |
|---|---|---|---|---|---|---|
| Antibody | CDR3L | CDR1H | CDR2H | CDR3H | LCVR | HCVR |
| V62.1 | 3 | 5 | 7 | 11 | 13 | 15 |

### Example 2: Affinity Maturation

The initial candidate as described above was then submitted to affinity maturation. Two Affinity Maturation Libraries were generated by CDR2H or CDR3L randomization, respectively. Each of these libraries was submitted to two rounds of high stringency selection. Selected Fabs were expressed individually, and clones with improved binding properties were retained. The best clones were reformatted as IgGs that were characterized for best CXCR4 binding affinity and selectivity as well as for best ability to prevent ligand induction of Ca-flux. As a final step, heavy and light chains of the most promising IgGs were recombined, and the resulting IgGs were again characterized as before.

In addition, CDR3H was matured by introduction of point mutations. Resulting mutated Fab fragments were characterized to identify the best CXCR4 binders.

The following matured antibody candidates were pursued further:

| | SEQ ID NO. | | | | | |
|---|---|---|---|---|---|---|
| Antibody* | CDR3L | CDR1H | CDR2H | CDR3H | LCVR | HCVR |
| V62.1-R108H | 3 | 5 | 7 | 12 | 13 | 16 |
| V62.1-R108H- | 4 | 5 | 8 | 12 | 14 | 17 |
| m43-m38 | | | | | | |
| V62.1- R108H- | 4 | 5 | 9 | 12 | 14 | 18 |
| m47-m38 | | | | | | |
| V62.1- R108H- | 3 | 6 | 10 | 12 | 13 | 19 |
| m80 | | | | | | |

*R108H refers to a point mutation in CDR3H (compare SEQ ID NO: 11 and 12), and m38, m43, m47 and m80 refer to particular selected CDR2H or CDR3L sequences, respectively.

As mentioned previously, all antibodies of the present invention share the same CDR1L (SEQ ID NO.1) and CDR2L (SEQ ID NO.2).

### Example 3: Synthesis of IgG Antibodies

The CHO/pTT Transient Transfection System from the Biotechnology Research Institute of the Canadian National Research Council (NRC-BRI) was used according to protocols provided by the NRC-BRI. See international patent application publication WO2009/137911 A1. More specifically, each IgG of interest was produced in CHO-3E7 cells co-transfected with pTT vectors expressing the light chain and the heavy chain of the IgG, respectively, using polyethylenimine (PEI) as a transfection reagent.

Cell medium containing IgGs was collected, and IgGs were purified on Protein A Plus Agarose (Pierce) using standard methodology. All purification procedures were performed using sterile, endotoxin-free solutions.

In the following examples, a Fab fragment of interest or an IgG antibody of interest is referred to as “test Fab”, “test antibody” or “test IgG”, as appropriate.

### Example 4: Binding to CXCR4-Expressing Cells

Binding of Fabs or IgGs to CXCR4-expressing cells was measured by a fluorescent flow cytometry-based assay. The cells were stained with:

(A) for Fabs—anti-c-Myc mouse antibody 9E10 Mab that binds to a tag present in the test Fab and then with secondary anti-mouse IgG phycoerythrin (PE)-conjugated antibody, or
(B) for IgGs—with anti-Human Fc PE-conjugated antibody.

As a control, cells that do not express CXCR4 or cells expressing other GPCR were used.

A typical protocol for the fluorescent flow cytometry-based assay was as follows. Ten microliter of a purified test IgG solution or buffer as a control were added to 10 microliter of a suspension containing approximately 30,000 Cf2-Th cells transfected to express human CXCR4. After incubation on ice for 40 min, cells were washed with FACS buffer (phosphate-buffered saline (PBS), pH7.4; 2% fetal calf serum, 0.1% sodium azide) to remove unbound antibodies. Ten microliter of a solution of phycoerythrin (PE)-conjugated mouse anti-human Fc monoclonal antibody (1/20 dilution; catalog number 12-4998-82, eBioscience Inc., San Diego, Calif.) were then added to the cells, and, after a 30-min incubation on ice, cells were washed twice and then formalin-fixed (FIX buffer: PBS, pH7.4; 0.5% formaldehyde). Fixed samples were analysed by fluorescent flow cytometry using a Guava-PCA96 instrument (EMD Millipore Chemicals, Merck KGaA, Darmstadt, Germany).

To determine an EC50 value, binding to the CXCR4-expressing cells was measured at different concentrations of test antibody. Duplicate or triplicate samples were analysed for each concentration. Titration curves were constructed based on the Mean Fluorescence Intensity (MFI) values provided by the instrument using a SoftMaxPro5 program (Molecular Devices Corp., Sunnyvale, Calif.).

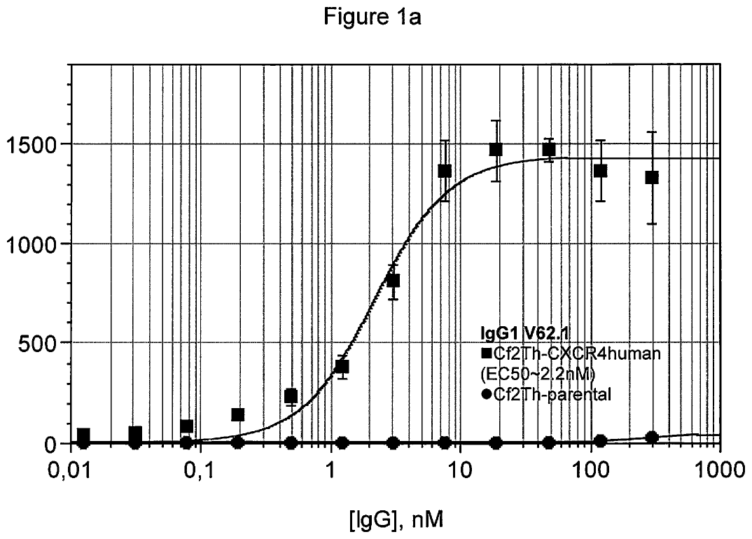

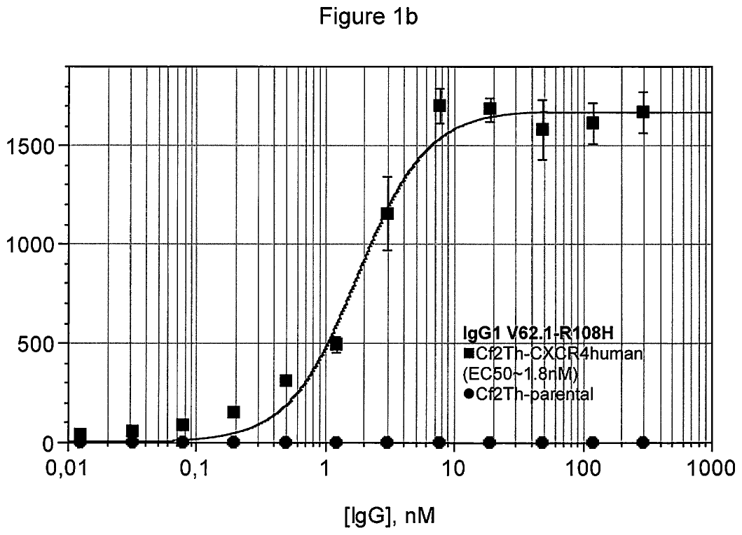

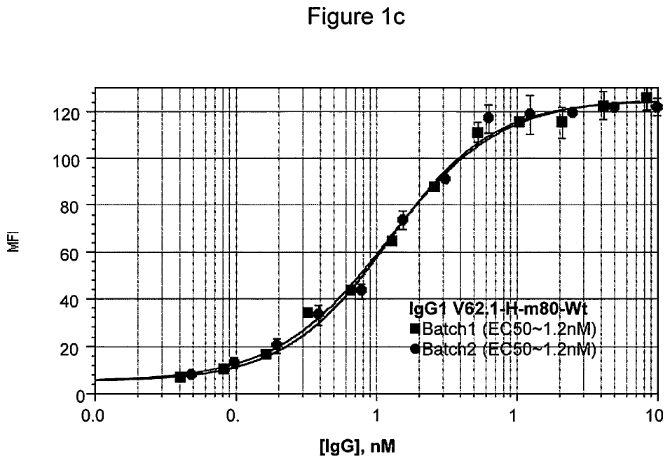

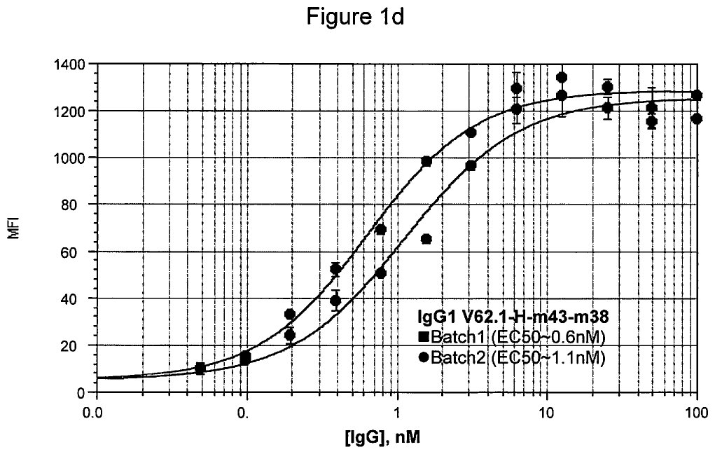

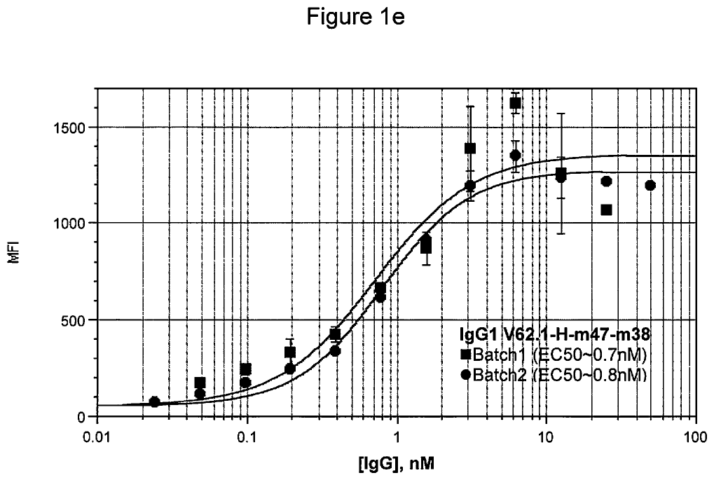

In some experiments, non-transfected Cf2-Th parental cells (ATCC® CRL-1430™) were used as negative controls, thereby establishing the specificity of the antibodies for CXCR4. In some other experiments, several batches of the same antibody candidate were tested in parallel. The dose response curves and EC50 results obtained with test antibodies in IgG1 format are presented in FIGS. 1a to e. All test IgG1 antibodies exhibited an EC50 well below 10 nM.

### Example 5: Binding to CXCR4-Expressing Human Lymphoma Cells

Binding of test IgGs to CXCR4-expressing human lymphoma cells (Ramos; RA1, ATCC® CRL-1596™) was measured by fluorescent flow cytometry-based assay. Tumor cell staining was conducted as follows: human Fcγ receptors of RA1 cells were saturated by incubation at 4° C. for 30 minutes in PBS containing 2% human serum and 0.5 mM EDTA. Cells were then incubated at 4° C. for one hour with test IgGs or a human isotypic IgG1 kappa control (Coger Sarl, Paris, France) (both antibody types at 10 μg/mL). The cells were washed with PBS and further incubated for one hour at 4° C. with a goat F(ab′)2 fragment anti-human IgG (H+L)-PE (Beckman Coulter). The cells were washed twice with PBS and fixed with 0.5% formaldehyde in PBS for analysis by flow cytometry. The data were acquired using an eleven-color flow cytometer (LSRII, BD Biosciences), and the analyses were performed with the FlowJo flow cytometry analysis software (Tree Star Inc., Ashland, Oreg.). The living cells were selected using the side scatter (SSC) and the forward scatter (FSC); 10,000 events were acquired for each analysis. MFI values were recorded using the PE channel.

| Antibody | MFI |
|---|---|
| V62.1 | 595 |
| V62.1-R108H | 429 |
| V62.1-R108H-m80 | 440 |
| V62.1-R108H-m43-m38 | 488 |
| V62.1-R108H-m47-m38 | 549 |
| IgG1k isotypic control | 42 |

MFI values well above that of the isotypic control indicate positive staining of the RA1 cells, which was observed for all test IgGs.

### Example 6: Specificity for CXCR4

Cells over-expressing different GPCRs other than CXCR4 and several lines transfected to over-express CXCR4 (R1610-hCXCR4, Cf2Th-hCXCR4 and CHO-hCXCR4) were compared. Cultures were incubated with a test IgG at 100 nM in FACS buffer for 40 min at 4° C. Thereafter, cells were washed twice, stained with anti-human-Fc antibody-PE conjugate (Jackson Immunoresearch Laboratories Inc., West Grove, Pa.), washed twice in FACS buffer and then transferred to FIX buffer. Fluorescence intensities were measured by GUAVA PCA-96 at 425V (in triplicate).

The expression of GPCRs other than CXCR4 was confirmed using commercially available antibodies (positive controls). For example, a commercial anti-CXCR1 antibody was used as a positive control for confirming the expression of CXCR1 on the CXCR1-transfected CHO cells.

A summary of the MFI data obtained is presented in the Table below.

| | | | V62.1- | | |
|---|---|---|---|---|---|
| | | | R108H- | V62.1 | |
| Antibody | | V62.1- | m47- | R108H | Positive |
| Cell line | V62.1 | R108H | m38 | m80 | controls |
| CHO-hCXCR1 | 4 | 4 | N/A | N/A | 1325 |
| hCXCR2 | 3 | 4 | N/A | N/A | 1830 |
| hCXCR3 | 3 | 3 | 2 | 2 | 500 |
| R1610- | 812 | 513 | N/A | N/A | 447 |
| hCXCR4 | | | | | |
| Cf2Th- | 1267 | 861 | 2350 | 3870 | 1006 |
| hCXCR4 | | | | | |
| CHO-hCXCR4 | 2053 | 1850 | N/A | N/A | 994 |
| hCXCR5 | 4 | 3 | 2 | 2 | 510 |
| hCXCR6 | 3 | 3 | 2 | 7 | 1050 |
| hCXCR7 | 3 | 4 | 2 | 2 | 300 |
| hCCR3 | 4 | 4 | 40 | 2 | 213 |
| hCCR4 | 3 | 3 | 2 | 2 | 506 |
| hCCR5 | 4 | 3 | 2 | 8 | 724 |
| hCCR6 | 3 | 3 | 2 | 10 | 1481 |
| hCCR7 | 3 | 4 | 2 | 25 | 465 |
| hCCR9 | 4 | 3 | 2 | 8 | 260 |
| hCCR10 | 4 | 3 | 2 | 10 | 3000 |
| Cf2Th | 3 | 3 | 2 | 8 | 1 |
| R1610 | 3 | 3 | 2 | 2 | 1 |
| CHO | 3 | 3 | 2 | 7 | 1 |

### Example 7: Inhibition of Ligand Binding to CXCR4

Inhibition of SDF-1alpha ligand binding was assayed by means of fluorescent flow cytometry using bacterially expressed SDF-1alpha containing an N-terminal FLAG tag. Cf2-Th cells transfected to express CXCR4 were incubated with a test IgG antibody (100 nM) for 30 min on ice. Thereafter, the FLAG-tagged ligand was added to a final concentration of 100 nM, and the cells were incubated for another 20 min. Subsequently, cells were washed, stained with an appropriate anti-FLAG tag PE-conjugated antibody and fixed with FIX buffer. In the control, no antibody was added. Then fluorescence was recorded. A decreased MFI value of cells that had been exposed to a test antibody (MFI_{WithAb}), as compared to the MFI of cells that had not been exposed to the IgG test antibody (MFI_{NoAb}) indicated a competition between the antibody and the ligand. Percent inhibition was defined as (MFI_{WithAb}/MFI_{NoAb})×100%. Similar MFI_{WithAb} and MFI_{NoAb} values indicated that the pre-bound test antibody failed to prevent binding of the tagged ligand to CXCR4 on the cell surface. Antibodies tested, V62.1 and V62.1R108H, were able to inhibit ligand binding by up to 96%.

### Example 8: Inhibition of SDF-1-Induced Calcium Flux

IC50 values were estimated based on data obtained from FLIPR calcium assays (Calcium-5 kit, Molecular Devices LLC, Sunnyvale, Calif.) on CXCR4-transfected Chem-1 cells (catalog no. HTS004C, EMD Millipore Chemicals). The cells were grown overnight at 37° C. and 5% CO₂. Before Ca-flux measurement, cells were starved in serum-free medium for 3 h at 37° C. and 5% CO₂. Dye was added to the cells which were then incubated for 30 min at 37° C. and 5% CO₂ in the presence of different concentrations of test IgG1 antibody. Control samples were prepared similarly, but no antibody was added. Thereafter, SDF-1alpha (R&D Systems) in TBS was added to the dye-loaded cells to a final concentration of 30 nM. Inhibition of the chemokine-induced increase in intracellular calcium concentration (Ca-flux) was calculated as follows:

$$Inhibition = ( 1 - \frac{[ \overline{I} ]}{[ \overline{C} ]} ) \times 100 \% ,$$

where [I]—means peak dye fluorescence (n=4) in inhibited samples, [C]—means peak dye fluorescence (n=18) in control samples.

Dose response curves were drawn and IC50 values calculated. IC50 represents the concentration of test IgG at which 50% inhibition of SDF-1alpha-induced calcium flux is observed. A summary of IC50 values determined for different test IgG is presented in the Table below:

| Test antibody | IC50 (nM): |
|---|---|
| V62.1 | 7.4 |
| V62.1-R108H | 6.8 |
| V62.1-R108H-m43-m38 | 3.8 |
| V62.1-R108H-m47-m38 | 5 |
| V62.1-R108H-m80-Wt | 7.5 |

All test antibodies significantly inhibited SDF-1alpha-induced calcium flux in CXCR4-expressing cells.

### Example 9: Inhibition of Chemotaxis/Cell Migration

Human U937 cells were grown in RPMI-1640 medium with 10% FCS, then washed twice and incubated in serum-free RPMI-1640 at 37° C. for 3 hours (5% CO₂). Starved 0937 cells were re-suspended in medium for chemotaxis (RPMI-1640 with 0.3% BSA) at 3*10\hat{ }5 cells per ml and
a) incubated for 30 min at room temperature (not-pre-treated positive control for assay);
b) pre-treated with AMD 3100 at 1 μM concentration (positive control for chemotaxis inhibition) for 30 min at room temperature; or
c) pre-treated with a test IgG at 100 nM concentration for 30 min at room temperature.

Respective not-pre-treated or pre-treated U937 cells were then placed into the top wells of a microchemotaxis chamber (15,000 cells per well). Bottom wells were supplemented as schematically presented in the table below.

| | (a) | (a1) | (b) | (c) |
|---|---|---|---|---|
| Top chamber | Non-pretreated | Non-pretreated | AMD 3100 Pre- | Test IgG pre- |
| | cells | cells | treated cells | treated cells |
| Bottom | Non- | SDF-1α (3 nM) | SDF-1α (3 nM) + | SDF-1α (3 nM) + |
| chamber | supplemented | | ADM 3100 (1 μM) | test IgG (100 nM) |
| | medium | | | |
| | Negative | Positive control | Positive control | |
| | control for | for chemotaxis | for chemotaxis | |
| | chemotaxis | | inhibition | |

A polycarbonate filter with a 8 μM pore diameter separated top and bottom chambers.

After incubation for one hour at 37° C. (5% CO₂), the cell suspensions were removed from the top wells, and the wells were washed once with PBS. Then the chamber was centrifuged for 4 min at 500 rpm, and migrated cells from bottom wells were transferred into wells of a V-shaped 96-well plate containing 50 μl PBS. The number of migrated cells in each well was determined using a Guava PCA-96 cytometer. All measurements were made in triplicates.

Maximal SDF-1 induced migration was calculated as the difference in the number of migrated cells between conditions (al) and (a) in the Table immediately above. The percentage of migration inhibition for a test antibody was then calculated by reference to this maximal migration.

The results for inhibition of SDF-1alpha-induced chemotaxis by different test IgGs are presented in the Table below.

| Tested conditions (All IgGs at 100 nM) | % Inhibition |
|---|---|
| SDF-1alpha, 3 nM + AMD3100, 1 μM | 98 |
| SDF-1alpha, 3 nM + V62.1 | 76 |
| SDF-1alpha, 3 nM + V62.1-R108H | 69 |
| SDF-1alpha, 3 nM + V62.1-R108H-m43-m38 | 77 |
| SDF-1alpha, 3 nM + V62.1-R108H-m47-m38 | 78 |
| SDF-1alpha, 3 nM + V62.1-R108H-m80 | 75 |

All test antibodies inhibited SDF-1alpha-induced chemotaxis by at least about 70%.

### Example 10: Antibody-Dependent Cell-Mediated Cytotoxicity (ADCC)

ADCC was measured with RA1 cells as the target cells (T) and using the CytoTox 96® Non-Radioactive Cytotoxicity Assay (Promega Corp., Fitchburg, Wis.)), which assay measures lactate dehydrogenase (LDH) release. A round-bottom 96-well culture plate was set up with the following control and experimental wells (100 microliter final volumes):

a. RA1 cells (for target cell spontaneous LDH release control)
b. RA1 cells (for target cell maximum LDH release control)
c. Culture medium (RPMI 1640 Medium (1X) without phenol red) used for volume correction control
d. Culture medium (for culture medium background control)
e. RA1 plus effector cells (E) (for target plus effector cell spontaneous LDH release control)
f. Experimental wells with effector and target cells (10⁵) with serial dilutions of each test IgG or without test IgG.

The plates were centrifuged at 1600 rpm for 2 minutes and incubated at 37° C. for 4 hours. One hour prior to supernatant harvest, 20 microliters of Lysis Solution (10×) were added to the conditions b) and c). The plates were centrifuged at 1600 rpm for 2 minutes, and 50 microliters of the supernatant from each well of the assay plates were transferred to corresponding wells of a flat-bottom 96-well enzymatic assay plate. Substrate Mix (50 microliters) was added to each well of the latter plate, and the plate (protected from light) was incubated for 30 minutes at room temperature. Finally, 50 microliters of Stop Solution were added to each well, and absorbance (OD values) was measured at 490 nm. Each condition was tested in triplicates.

Preliminary experiments had been conducted in order to determine the optimal E:T ratio. The data shown below were obtained at a 20:1 E:T ratio with 10⁵ RA1 target cells. The effector cells used in this study were prepared as follows: human peripheral blood mononuclear cells (PBMCs) obtained from a healthy donor were isolated by means of density gradient centrifugation using Lymphocyte Separation Medium (ref. J15-004, Invitrogen Corp., Carlsbad, Calif.). PBMCs at 2×10⁶ per mL were cultured for two days in RPMI 1640 with 10% FCS, penicillin/streptomycin and 100 units per mL of human recombinant IL-2 (obtained from Roussel-Uclaf) in a 37° C. humidified incubator (5% CO₂).

ADCC percentages obtained at different concentrations of test IgGs were calculated with the formula % ADCC=(f−e)/(b−a)×100, i.e., % ADCC=(OD of Target+Effector cells +/−Test Mab −OD of Spontaneous Release of Target+Effector cells)/(OD Maximal Release Target cells −OD Spontaneous Release of Target cells)×100.

The data shown in the Table below demonstrate that all test antibodies induced significant cell-mediated cytotoxicity.

| | ADCC percentage | | | | |
|---|---|---|---|---|---|
| | 1 | 0.1 | 0.01 | 0.001 | 0.0001 |
| IgGs | μg/mL | μg/mL | μg/mL | μg/mL | μg/mL |
| Rituximab | 60 | 46 | 27 | 8 | 3 |
| IgG1 V62.1 R108H | 59 | 57 | 23 | 15 | 8 |
| IgG1 62.1-R108H-m80 | 55 | 59 | 45 | 16 | 9 |
| IgG1 62.1-R108H-m47- | 55 | 51 | 50 | 36 | 14 |
| m38 | | | | | |
| IgG1 62.1-R108H-m43- | 54 | 42 | 40 | 19 | 8 |
| m38 | | | | | |
| IgG V62.1 | 45 | 44 | 45 | 28 | 8 |
| IgG1k | 5 | 0 | −4 | −5 | −7 |

### Example 11: Anti-Tumor Activity in a SCID/RA1 Xenograft Model

The therapeutic effect of test antibodies was evaluated in an animal model of Burkitt's lymphoma. In the model used, systemic cancer in SCID mice causes hind limb paralysis and infiltrates all major organs.

Forty-eight hours before tumor cell injection, female SCID mice (7-9 weeks old, weighing 17-22 g) were irradiated with a γ-source (1.8 Gy, ⁶⁰Co). At D0, one million RA1 cells (B lymphocyte-type cell line established from a patient with American-type Burkitt lymphoma) suspended in 200 μl of RPMI 1640 were intravenously injected into the caudal vein of the mice. The tumor bearing mice were distributed on D4 into 10 groups of 10 mice each based on body weight using Vivo Manager® software (Biosystemes, Dijon, France). Mean body weights were not statistically different from one group to another (mean body weight: 19.3±1.4 g). Treatment started on D4: mice were administered intravenously a 5 mg/kg or a 10 mg/kg dose of test antibody on days 4, 8, 12, 16, 20 and 24. The vehicle for injection was 10 mM Na-Citrate, 150 mM NaCl, 50 mM Arginine (pH 5.5). Body weights were measured and recorded twice weekly. Mean survival time was calculated for each group as the mean of the day of death, and median survival time was calculated for each group as the median of the day of death. The efficacy of each test antibody was judged by the increased life span value (ILS). ILS % was expressed as follows: ILS %=[(T-C)/C]×100. T was the median of the survival times of animals treated with each test antibody, and C was the median survival time of control animals treated with vehicle. The experiment was terminated 81 days after tumor injection.

All control mice treated with vehicle or Xolair® (isotypic control) died of disseminated disease with severe weight loss or were terminated because they were moribund within four weeks after tumor cell inoculation.

Repeated treatment with antibodies V62.1 or V62.1-R108H led to a 2-fold increase in survival rate in comparison with control mice (p<0.001). In addition, the mice treated with CD20 antibody Mabthera®, used as positive control, lived significantly longer than control mice (p<0.001). These data indicated that test antibodies V62.1 and V62.1-R108H could effectively target and suppress RA1 cell proliferation in this highly aggressive xenograft model. Detailed results are shown in the Table below.

| | Mice alive at | | | |
|---|---|---|---|---|
| | the end of the | Median | Mean | % |
| | study = | Survival | Survival | Increased |
| Groups | day 81 | (days) | (days) | life span |
| Vehicle (IV, Q4D × 6) | 0/10 | 27 | 28 | — |
| V62.1 (10 mg/kg, IV, | 0/10 | 61 | 61 | 126 |
| Q4D × 6) | | | | |
| V62.1-R108H | 1/10 | 59 | 60 | 119 |
| (10 mg/kg, IV, | | | | |
| Q4D × 6) | | | | |
| Mabthera (10 mg/kg, | 0/10 | 62 | 63 | 130 |
| IV, Q4D × 6) | | | | |
| Xolair (10 mg/kg, | 0/10 | 27 | 29 | 0 |
| IV, Q4D × 6) | | | | |

### Example 12: Anti-Metastatic Activity in a Human Breast Cancer Xenograft Model

The aim of this experiment was to evaluate the efficacy of four test antibodies in a breast carcinoma metastasis model in which MDA-MB-231/Luc cells (cat. no. AKR-231, Cell Biolabs, Inc, San Diego, Calif.) were implanted intravenously in BALB/c nude mice. The MDA-MB-231/Luc cell line is a luciferase-expressing subline derived from the MDA-MB-231 human breast cancer cell line. MDA-MB-231/Luc cells produce experimental metastasis in the lung. Transendothelial MDA-MB-231 cancer cell migration as well as vascular permeability was known to depend on SDF1/CXCR4 signaling (Lee et al. (2004) Mol. Cancer Res. 2: 327).

The study consisted of 7 experimental groups each containing 12 female BALB/c nude mice. On day −1, animals were randomized based on body weight. The mean body weight of each group was not statistically different from the others by variance analysis. On day 0, 2×10⁶ MDA-MB-231/Luc cells in 100 μl 0.9% NaCl were implanted intravenously into all participating animals. Metastatic growth was assessed on days 2, 9, 15, 24, 28, 31, 35 and 38 using in vivo bioluminescence imaging. Animal weights were measured every other day (Monday, Wednesday and Friday). Animals of Groups 2-5 were intravenously administered 10 mg/kg of test antibody on days −1, 3, 7, 11, 15, and 19 (Q4D×6), and group 1 received vehicle (10 mM Na-Citrate, 150 mM NaCl, 50 mM Arginine, pH 5.5). Antibody doses were calculated based on the latest body weight measurements.

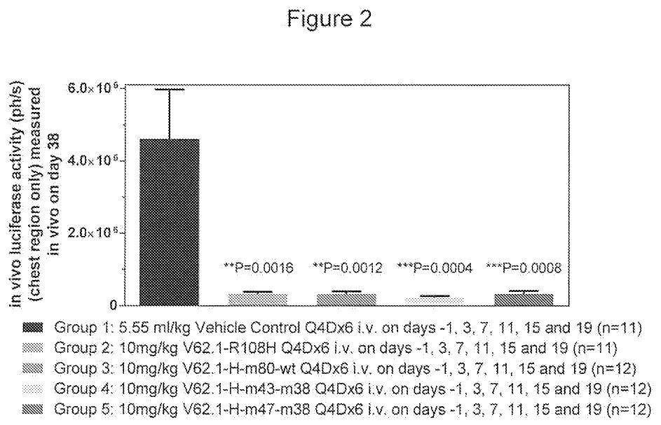

Total luciferase activity in the chest region at the end of the study (at day 38) is shown in FIG. 2. All test antibodies significantly inhibited tumor cell growth in the chest region.

Recitation of ranges of values herein is merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e. g., all exact exemplary values provided with respect to a particular factor or measurement can be considered to also provide a corresponding approximate measurement, modified by “about,” where appropriate).

The use of any and all examples, or exemplary language (e.g., “such as”) provided herein is intended merely to better illuminate the invention and does not pose a limitation on the scope of the invention unless otherwise indicated.

The citation and incorporation of patent documents herein is done for convenience only and does not reflect any view of the validity, patentability and/or enforceability of such patent documents. The description herein of any aspect or embodiment of the invention using terms such as reference to an element or elements is intended to provide support for a similar aspect or embodiment of the invention that “consists of′,” “consists essentially of” or “substantially comprises” that particular element or elements, unless otherwise stated or clearly contradicted by context (e. g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the essence or scope of the appended claims.

1. A monospecific antibody or binding fragment, comprising (i) a light chain variable region comprising the amino acid sequence of SEQ ID NO: 13 or 14, or an amino acid sequence that differs from said amino acid sequences by one or more conservative modifications and (ii) a heavy chain variable region comprising the amino acid sequence of SEQ ID NO: 15, 16, 17, 18 or 19, or an amino acid sequence that differs from said amino acid sequences by one or more conservative modifications.

2. A monospecific antibody or binding fragment having (i) a variable light chain comprising a CDR1L having the sequence of SEQ ID NO: 1 or an amino acid sequence that differs from said amino acid sequence by one or more conservative modifications; a CDR2L having the sequence of SEQ ID NO: 2 or an amino acid sequence that differs from said amino acid sequence by one or more conservative modifications; and a CDR3L having the sequence of SEQ ID NO: 3 or 4, or an amino acid sequence that differs from said amino acid sequences by one or more conservative modifications; and (ii) a variable heavy chain comprising a CDR1H having the sequence of SEQ ID NO: 5 or 6, or an amino acid sequence that differs from said amino acid sequences by one or more conservative modifications; a CDR2H having the sequence of SEQ ID NO: 7, 8, 9 or 10, or an amino acid sequence that differs from said amino acid sequences by one or more conservative modifications; and a CDR3H having the sequence of SEQ ID NO: 11 or 12, or an amino acid sequence that differs from said amino acid sequences by one or more conservative modifications.

3. The monospecific antibody or binding fragment of claim 1 or 2, whereby the monospecific antibody or binding fragment is a human engineered antibody or binding fragment.

4. The monospecific antibody or binding fragment according to any one of claims 1 to 3, whereby the monospecific antibody or binding fragment is of the IgG1 isotype.

5. The monospecific antibody or binding fragment according to any one of claims 1 to 4 for use in treatment of a cancer expressing CXCR4.

6. The monospecific antibody or binding fragment as defined in any one of claims 1 to 4 for use in treating Burkitt's lymphoma.

7. The monospecific antibody or binding fragment as defined in any one of claims 1 to 4 for use in treating breast cancer.

8. The use of any one of claims 5 to 7, wherein the subject treated is a human subject.

9. A method of detecting CXCR4-expressing cancer cells in a mammalian subject, the method comprises (i) taking from the subject a biopsy or fluid sample containing cancer cells and using the monospecific antibody or binding fragment of any of claims 1 to 4 in an immunochemical or immunohistochemical assay that detects expression of CXCR4 in the cancer cells or (ii) parenterally administering to the subject a radiolabeled monospecific antibody or binding fragment of any of claims 1 to 4 and detecting the antibody or binding fragment in the subject by immunoscintigraphy.

10. The method of claim 9, wherein the mammalian subject is a human subject.

11. A pharmaceutical composition comprising a therapeutically effective amount of a monospecific antibody or binding fragment according to any of claims 1 to 4 and a pharmaceutically acceptable excipient.

12. A pharmaceutical composition for parenteral administration to a subject, comprising a therapeutically effective amount of a monospecific antibody or binding fragment according to any of claims 1 to 4, a parenterally acceptable diluent and, optionally, a pharmaceutically acceptable excipient.

13. A diagnostic kit comprising a monospecific antibody or binding fragment according to any of claims 1 to 4.

14. A polynucleotide encoding a monospecific antibody or binding fragment according to any of claims 1 to 4.


SEQUENCE LISTING (38 sequences)

SEQ ID NO: 1 - protein, 7 aa, Artificial Sequence
  light chain CDR1L
SSYLAWY

SEQ ID NO: 2 - protein, 10 aa, Artificial Sequence
  light chain CDR2L
LLIYGASSRA

SEQ ID NO: 3 - protein, 9 aa, Artificial Sequence
  light chain CDR3L, variant 1
QQSDYSYPF

SEQ ID NO: 4 - protein, 8 aa, Artificial Sequence
  light chain CDR3L, variant 2
QQWSSDSV

SEQ ID NO: 5 - protein, 6 aa, Artificial Sequence
  heavy chain CDR1H, variant 1
SSYAMS

SEQ ID NO: 6 - protein, 6 aa, Artificial Sequence
  heavy chain CDR1H, variant 2
SPYSIT

SEQ ID NO: 7 - protein, 13 aa, Artificial Sequence
  heavy chain CDR2H, variant 1
WVSAISGSGGSTY

SEQ ID NO: 8 - protein, 14 aa, Artificial Sequence
  heavy chain CDR2H, variant 2
WVSAISGSGGGSTR

SEQ ID NO: 9 - protein, 12 aa, Artificial Sequence
  heavy chain CDR2H, variant 3
WVSAISGSGGTR

SEQ ID NO: 10 - protein, 13 aa, Artificial Sequence
  heavy chain CDR2H, variant 4
WVSQINSYNGKTY

SEQ ID NO: 11 - protein, 23 aa, Artificial Sequence
  heavy chain CDR3H, variant 1
ARQGKTRVYGYRWHGYGSTHALD

SEQ ID NO: 12 - protein, 23 aa, Artificial Sequence
  heavy chain CDR3H, variant 2
ARQGKTRVYGYHWHGYGSTHALD

SEQ ID NO: 13 - protein, 111 aa, Artificial Sequence
  light chain variable region LCVR, variant 1
EIVLTQSPGTLSLSPGERATLSCRASQSVSSSYLAWYQQKPGQAPRLLIYGASSRATGIPDRFSGSGSGTDFTLTISRLEPEDFAVYYCQQSDYSYPFTFGQGTKVEIKRT

SEQ ID NO: 14 - protein, 110 aa, Artificial Sequence
  light chain variable region LCVR, variant 2
EIVLTQSPGTLSLSPGERATLSCRASQSVSSSYLAWYQQKPGQAPRLLIYGASSRATGIPDRFSGSGSGTDFTLTISRLEPEDFAVYYCQQWSSDSVTFGQGTKVEIKRT

SEQ ID NO: 15 - protein, 131 aa, Artificial Sequence
  heavy chain variable region HCVR, variant 1
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQGKTRVYGYRWHGYGSTHALDYWGQGTLVTVSS

SEQ ID NO: 16 - protein, 131 aa, Artificial Sequence
  heavy chain variable region HCVR, variant 2
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQGKTRVYGYHWHGYGSTHALDYWGQGTLVTVSS

SEQ ID NO: 17 - protein, 132 aa, Artificial Sequence
  heavy chain variable region HCVR, variant 3
EVQLLESGGGLVQPGGSLRLSCAASGFTFSSYAMSWVRQAPGKGLEWVSAISGSGGGSTRYADSVKGRFTISRDNSKNTLYLQMNSLRAEDTAVYYCARQGKTRVYGYHWHGYGSTHALDYWGQGTLVTVSS

SEQ ID NO: 18 - protein, 130 aa, Artificial Sequence
  heavy chain variable region HCVR, variant 4
EVQLLESGGGLVQPGGSLRLSCAASGFTFSSYAMSWVRQAPGKGLEWVSAISGSGGTRYADSVKGRFTISRDNSKNTLYLQMNSLRAEDTAVYYCARQGKTRVYGYHWHGYGSTHALDYWGQGTLVTVSS

SEQ ID NO: 19 - protein, 131 aa, Artificial Sequence
  heavy chain variable region HCVR, variant 5
EVQLLESGGGLVQPGGSLRLSCAASGFTISPYSITWVRQAPGKGLEWVSQINSYNGKTYYADSVKGRFTISRDNSKNTLYLQMNSLRAEDTAVYYCARQGKTRVYGYHWHGYGSTHALDYWGQGTLVTVSS

SEQ ID NO: 20 - DNA, 21 bp, Artificial Sequence
  CDR1L
tcctcttacctggcctggtat

SEQ ID NO: 21 - DNA, 30 bp, Artificial Sequence
  CDR2L
ctgctgatctatggcgcctcttctagagcc

SEQ ID NO: 22 - DNA, 27 bp, Artificial Sequence
  CDR3L, variant 1
cagcagtccgactactcctaccccttc

SEQ ID NO: 23 - DNA, 24 bp, Artificial Sequence
  CDR3L, variant 2
cagcagtggtcctccgactccgtg

SEQ ID NO: 24 - DNA, 18 bp, Artificial Sequence
  CDR1H, variant 1
tccagctacgccatgtcc

SEQ ID NO: 25 - DNA, 18 bp, Artificial Sequence
  CDR1H, variant 2
tccccctactctatcacc

SEQ ID NO: 26 - DNA, 39 bp, Artificial Sequence
  CDR2H, variant 1
tgggtgtccgccatctctggctctggcggctctacctac

SEQ ID NO: 27 - DNA, 42 bp, Artificial Sequence
  CRD2H, variant 2
tgggtgtccgccatcagtggctctggcggaggctctaccaga

SEQ ID NO: 28 - DNA, 36 bp, Artificial Sequence
  CDR2H, variant 3
tgggtgtccgccatctccggctccggcggcaccaga

SEQ ID NO: 29 - DNA, 39 bp, Artificial Sequence
  CDR2H, variant 4
tgggtgtcccagatcaactcctacaacggcaagacctac

SEQ ID NO: 30 - DNA, 69 bp, Artificial Sequence
  CDR3H, variant 1
gctagacagggcaagacccgggtgtacggctaccgttggcacggctatggctctacccacgccctggat

SEQ ID NO: 31 - DNA, 69 bp, Artificial Sequence
  CDR3H, variant 2
gctagacagggcaagacccgggtgtacggctaccactggcacggctatggctctacccacgccctggat

SEQ ID NO: 32 - DNA, 333 bp, Artificial Sequence
  LCVR, variant 1
gagatcgtgctgacccagtctcctggcaccctgtctctgagccctggcgagagagctaccctgtcctgcagagcctcccagtccgtgtcctcctcttacctggcctggtatcagcagaagcccggccaggctccccggctgctgatctatggcgcctcttctagagccaccggcatccccgacagattctccggctctggctctggcaccgacttcaccctgaccatctcccggctggaacccgaggacttcgccgtgtactactgccagcagtccgactactcctaccccttcaccttcggccagggcaccaaggtggaaatcaagcggacc

SEQ ID NO: 33 - DNA, 330 bp, Artificial Sequence
  LCVR, variant 2
gagatcgtgctgacccagtcccccggcaccctgtctctgagccctggcgagagagccaccctgtcctgcagagcctcccagtccgtgtcctcctcctacctggcctggtatcagcagaagcccggccaggcccctcggctgctgatctacggcgcctcttccagagccaccggcatccctgaccggttctccggctctggctccggcaccgacttcaccctgaccatctcccggctggaacccgaggacttcgccgtgtactactgccagcagtggtcctccgactccgtgaccttcggccagggcaccaaggtggaaatcaagcggacc

SEQ ID NO: 34 - DNA, 393 bp, Artificial Sequence
  HCVR, variant 1
gaagtgcagctgctggaatctggcggcggactggtgcagcctggcggatctctgagactgtcttgtgccgcctccggcttcaccttctccagctacgccatgtcctgggtgcgacaggctcctggcaagggcctggaatgggtgtccgccatctctggctctggcggctctacctactacgccgactccgtgaagggccggttcaccatctcccgggacaactccaagaacaccctgtacctgcagatgaactccctgcgggccgaggacaccgccgtgtactactgtgctagacagggcaagacccgggtgtacggctaccgttggcacggctatggctctacccacgccctggattattggggccagggcaccctcgtgaccgtgtcctct

SEQ ID NO: 35 - DNA, 393 bp, Artificial Sequence
  HCVR, variant 2
gaagtgcagctgctggaatctggcggcggactggtgcagcctggcggatctctgagactgtcttgtgccgcctccggcttcaccttctccagctacgccatgtcctgggtgcgacaggctcctggcaagggcctggaatgggtgtccgccatctctggctctggcggctctacctactacgccgactccgtgaagggccggttcaccatctcccgggacaactccaagaacaccctgtacctgcagatgaactccctgcgggccgaggacaccgccgtgtactactgtgctagacagggcaagacccgggtgtacggctaccactggcacggctatggctctacccacgccctggattattggggccagggcaccctcgtgaccgtgtcctct

SEQ ID NO: 36 - DNA, 396 bp, Artificial Sequence
  HCVR, variant 3
gaagtgcagctgctggaatctggcggcggactggtgcagcctggcggatctctgagactgtcttgtgccgcctccggcttcaccttctccagctacgccatgtcctgggtgcgacaggctcctggcaagggcctggaatgggtgtccgccatcagtggctctggcggaggctctaccagatacgccgactctgtgaagggccggttcaccatctcccgggacaactccaagaacaccctgtacctgcagatgaactccctgcgggccgaggacaccgccgtgtactactgtgctagacagggcaagacccgggtgtacggctaccactggcacggctacggctctacccacgccctggattattggggccagggcaccctcgtgaccgtgtcctct

SEQ ID NO: 37 - DNA, 390 bp, Artificial Sequence
  HCVR, variant 4
gaggtgcagctgctggaatccggcggaggactggtgcagcctggcggctccctgagactgtcttgcgccgcctccggcttcaccttctccagctacgccatgtcctgggtgcgacaggcccctggcaagggcctggaatgggtgtccgccatctccggctccggcggcaccagatacgccgactctgtgaagggccggttcaccatctcccgggacaactccaagaacaccctgtacctgcagatgaactccctgcgggccgaggacaccgccgtgtactactgcgccagacagggcaagacccgggtgtacggctaccactggcacggctacggctccacccacgccctggattattggggccagggcaccctggtgacagtgtcctcc

SEQ ID NO: 38 - DNA, 393 bp, Artificial Sequence; identical to SEQ ID NO: 35
  HCVR, variant 5